(19) United States
(12) Patent Application Publication
(10) Pub. No.: US 2010/0268072 A1
(43) Pub. Date: Oct. 21, 2010

(54) METHOD AND APPARATUS FOR POSITIONAL TRACKING OF THERAPEUTIC ULTRASOUND TRANSDUCER

(75) Inventors: Christopher Stephen Hall, Hopewell Junction, NY (US); Chien Ting Chin, Tarrytown, NY (US); Balasundara I. Raju, Tarrytown, NY (US); William Tao Shi, Briarcliff Manor, NY (US); Guy Shechter, Briarcliff Manor, NY (US)

Correspondence Address: PHILIPS INTELLECTUAL PROPERTY & STANDARDS, P.O. BOX 3001, BRIARCLIFF MANOR, NY, 10510, US

(73) Assignee: KONINKLIJKE PHILIPS ELECTRONICS N.V., EINDHOVEN (NL)

(21) Appl. No.: 12/741,831

(22) PCT Filed: Nov. 13, 2008
(86) PCT No.: PCT/IB2008/054767

§ 371 Date: May 25, 2010

Related U.S. Application Data
(60) Provisional application No. 60/988,110, filed on Nov. 15, 2007.

(51) Int. Cl. A61B 5/05; A61N 7/00

(52) U.S. Cl. 600/427

(57) ABSTRACT

A therapeutic ultrasound tracking system (10) includes a first and second plurality of tracking elements ((14, 16, 18),(20,22,24)), a tracking generator (26), and a system controller (28). The first plurality of tracking elements (14, 16, 18) is disposed in a first position and orientation with respect to each another and with respect to an energy emission surface (30) of a therapeutic ultrasound probe (12). The second plurality of tracking elements (20,22,24) is adapted to be coupled to a patient (42) in a second position and orientation with respect to each other and with respect to a target region of the patient which is to receive a therapeutic treatment. The tracking generator (26) emits tracking energy for use in connection with the first and second plurality of tracking elements. The system controller (28) detects whether the probe (12) is positioned within an allowable position and orientation with respect to the target region, and (i) responsive to detecting the probe being within the allowable position and orientation, the system controller enables an energization of the probe to perform one of (i)(a) commence the therapeutic treatment and (i)(b) continue the therapeutic treatment, and (ii) responsive to not detecting the probe being within the allowable position and orientation, the system controller disables the energization of the probe for the therapeutic treatment.

The present embodiments relate generally to medical systems and more particularly, to a method and system apparatus for positional tracking of a therapeutic ultrasound transducer.

Current treatment of pathological tissue with ultrasound is often not performed effectively because the delineation of the disease with ultrasound imaging is not as clear with another imaging modality such as computer tomography (CT).

However, ultrasound is becoming a more desirable approach for specific therapeutic interventions. In particular, the use of high intensity focused ultrasound is currently being used as an approach for thermal therapeutic intervention for uterine fibroids and has been examined for possible uses in the treatment of liver, brain, and other cancerous lesions. In addition, ultrasound has also been the subject of much research as a means of mediating clot dissolution (sonothrombolysis), drug delivery, and gene therapy. The use of ultrasound in all of these applications is desirable because it allows the non-invasive treatment of deep tissues with little or no effect on overlying organs. Ultrasound has also been shown to increase the efficacy of existing medical treatments such as the use of tissue plasminogen activator (tPA) in clot dissolution, expression of proteins in gene therapy, and increased delivery of drugs in site-targeted therapies.

However, for many medical conditions, the diagnosis and treatment planning is done using imaging modalities other than ultrasound, such as computerized tomography (CT), magnetic resonance imaging (MRI), SPECT, and positron emission tomography (PET). In addition, these systems are used independent of the methods used for therapy. Accordingly, an improved method and system for overcoming the problems in the art is desired.

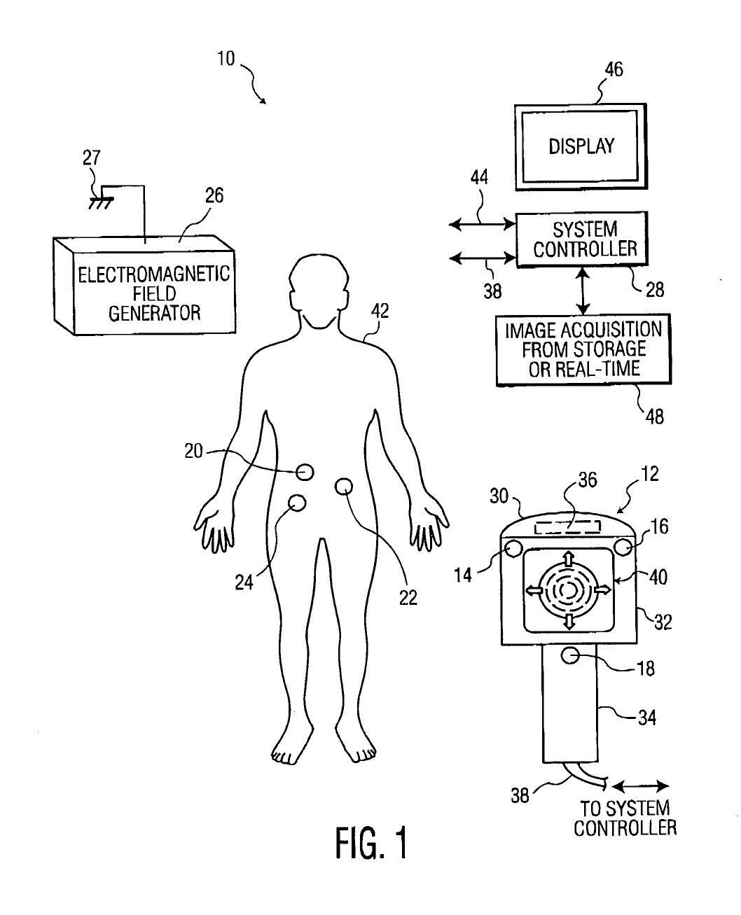

FIG. 1 is a partial block diagram view of a system for positional tracking of a therapeutic ultrasound probe according to an embodiment of the present disclosure;

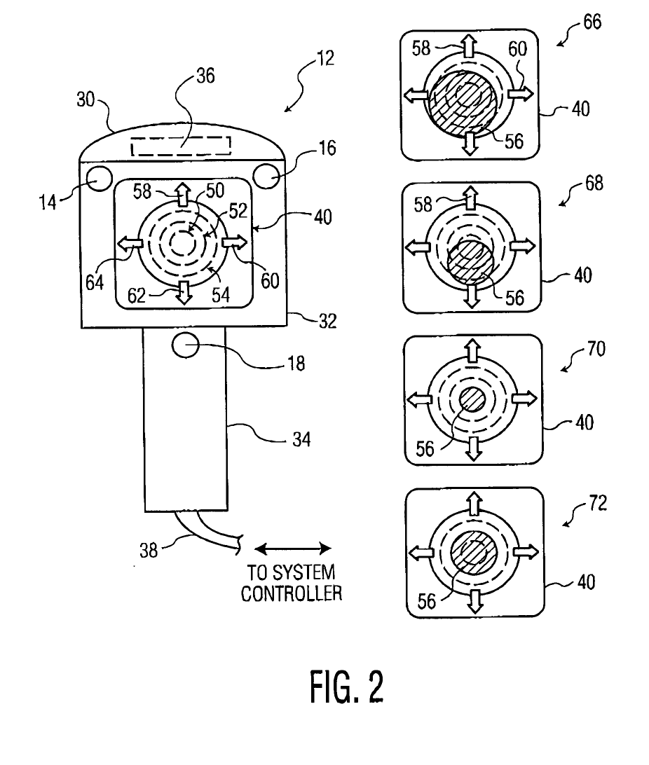

FIG. 2 is a schematic representation view of the therapeutic ultrasound probe of FIG. 1 shown in greater detail for use with the system for positional tracking according to an embodiment of the present disclosure;

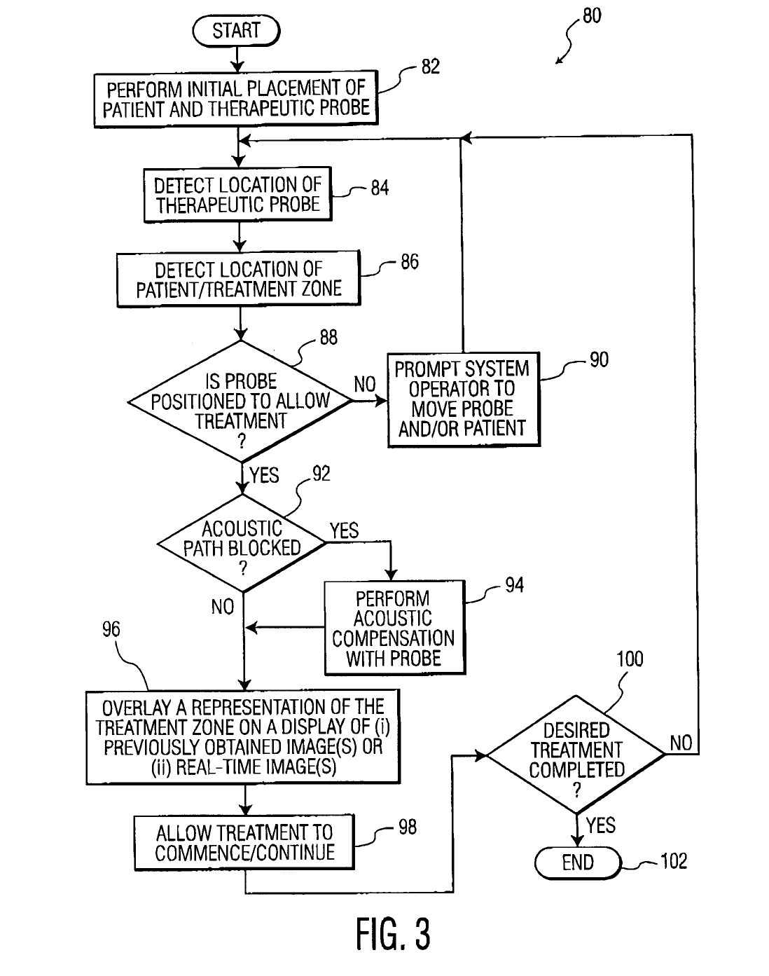

FIG. 3 is a flow diagram view illustrating positional tracking of a therapeutic ultrasound probe of the system and method according to an embodiment of the present disclosure; and

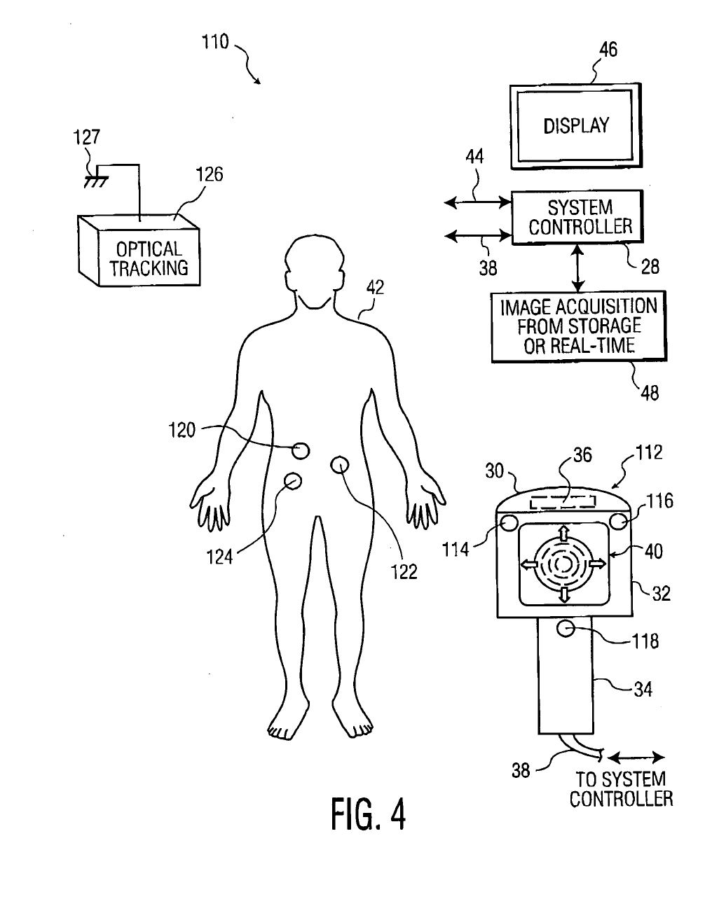

FIG. 4 is a partial block diagram view of a system for positional tracking of a therapeutic ultrasound probe according to another embodiment of the present disclosure.

In the figures, like reference numerals refer to like elements. In addition, it is to be noted that the figures may not be drawn to scale.

According to one embodiment, a method and apparatus for positional tracking of a therapeutic ultrasound transducer or probe comprises a positioning system and therapeutic ultrasound probe configured for improving therapeutic outcomes. The method and system apparatus advantageously render it possible to use one imaging modality, such as CT, to diagnose and plan a therapeutic intervention, and then apply ultrasound therapy to the desired spatial location. The efficacy of the treatment is thereby advantageously improved. In addition, the treatment can be localized to a specific region of interest (e.g., treatment region) with minimal effect on surrounding tissues. In one embodiment, a position-tracking system and an ultrasound therapeutic probe are registered through proper placement of a plurality of sensors into the same coordinate system.

Technology for tracking of an object in three-dimensional space is known and includes, for example, electromagnetic tracking, infrared (light) tracking, and mechanical positioning systems. These tracking technologies allow sub-millimeter accuracies for detecting the position of an object. Accordingly, for purpose of simplicity of discussion, only those aspects of tracking technology necessary for understanding the embodiments of the present disclosure are discussed herein.

According to another embodiment of the present disclosure, treatment of pathological tissue with high intensity focused ultrasound can be performed effectively despite the delineation of the disease with ultrasound imaging not being as clear with another imaging modality such as CT. One example is the treatment of a patient presenting with an occlusive stroke. The patient is first imaged with a CT scan to confirm the presence of the blood clot. The CT scan is then registered to the patient's physical body, and the same registration system is used to guide a sonothrombolytic treatment with a high-intensity focused ultrasound probe of the positional tracking therapeutic ultrasound system according to the embodiments of the present disclosure. As a result, the therapeutic treatment's effectiveness through accurate placement of adequate acoustic energy into the clotted vessel is advantageously improved. Similar examples of therapeutic treatment include drug delivery, high intensity focused ultrasound (HIFU) ablation, and gene delivery. Therapeutic treatment can also include cosmetic uses of ultrasound, for example, fat treatment. In the latter example, MR might be a good modality to use for obtaining a delineation of the fat tissue.

In one embodiment, gene therapy can comprise one or more of the delivery of foreign DNA, RNA, RNAi, enzymes, proteins, siRNA, or micro RNA. The use of ultrasound as a therapeutic technology in areas varying from high intensity focused ultrasound for thermal ablation to the use for drug and gene delivery is becoming a clinical reality. The diagnostic imaging system (e.g. CT, MRI etc.) used for delineating the spatial location of the pathological tissue to be treated is acquired independently from the treatment regimen. With the advantageous embodiments of the present disclosure, ultrasonic therapeutic exposures can be properly applied to the specific region of interest by a method of spatially registering the application of therapeutic, focused ultrasound to the diagnostic image and to the treatment region of the patient.

The method and apparatus of the present disclosure are directed to an approach for using external position tracking systems to maintain registration between the treatment region of the patient and the ultrasonic therapy probe. For example, the system apparatus comprises a position-tracking system and an ultrasound therapeutic transducer registered through proper placement of sensors into the same coordinate system, to be discussed further herein.

In one exemplary embodiment, an electromagnetic tracking system is placed on a tabletop on which the patient is lying immobile. The tabletop is registered exactly in the coordinate frame of an imaging system (CT, MRI, SPECT, PET, etc.). Coils (sensors) are placed at one or more locations on the ultrasound therapeutic transducer in such a way that the electromagnetic tracking system can calculate the three-dimensional position and orientation of the therapy transducer with respect to its own coordinate frame. The ultrasound therapy probe is now tracked in the patient's coordinate frame and can be focused on the pathological area to effect treatment. In a second exemplary embodiment similar to the preceding embodiment, the tracking system includes an infra-red tracking system and the sensors comprise light emitting diodes.

In a third exemplary embodiment similar to the first preceding embodiment, the tracking system includes a plurality of coils capable of providing a measure of a magnetic field. In addition, another plurality of coils is coupled to the therapy transducer. For example, the coils can be coupled to the therapy transducer via suitable mechanical attachment or the like. The measure of the magnetic field provides an indication of position relative to a magnetic resonance imaging (MRI) system. In a fourth exemplary embodiment, similar to the first and second exemplary embodiments, sensors are attached to a fixed frame, for example, a headset in which the ultrasound transducer may be secured. The headset may be initially placed according to anatomical landmarks or through co-registration with a previous imaging data set. After placement, the tracking system is used to measure small-scale movements.

The embodiments of the present disclosure advantageously provide a method and system apparatus for performing one of more of: (i) Ultrasound mediated drug delivery; (ii) Ultrasound mediated gene delivery; (iii) Ultrasound thermal therapies; (iv) High intensity focused ultrasound ablation, (v) Sonothrombolysis, and (vi) Ultrasound cosmetic surgery including fat and scar/wrinkle removal.

FIG. 1 is a partial block diagram view of a system 10 for positional tracking of a handheld therapeutic ultrasound probe 12 according to an embodiment of the present disclosure. The therapeutic ultrasound tracking system 10 comprises a first plurality of tracking elements (14,16,18), a second plurality of tracking elements (20,22,24), a tracking generator 26, and a system controller 28. The first plurality of tracking elements (14,16,18) is disposed in a first position and orientation with respect to each another and with respect to an energy emission surface 30 of the handheld therapeutic ultrasound probe 12. Therapeutic ultrasound probe 12 can include any suitable handheld ultrasound probe or other probe that can be configured for implementing the embodiments of the present disclosure and for carrying out the requirements of a given ultrasound therapy. Therapeutic ultrasound probe 12 includes a housing portion 32 and a handle portion 34, which can be formed integral with one another. An ultrasound transducer 36 is disposed within housing portion 32, proximate surface 30, for emission of desired ultrasound energy. Various electrical power and signal lines, collectively represented by the feature indicated by reference numeral 38, are coupled between components of the therapeutic ultrasound probe 12 and system controller 28, for example, as appropriate, for carrying out various functions and steps described herein. A tracking guide 40 coupled to therapeutic ultrasound probe 12 is also illustrated, as will be discussed further herein with reference to FIG. 2.

The second plurality of tracking elements (20,22,24) is adapted to be coupled to a patient 42 in a second position and orientation with respect to each other and with respect to a target region of the patient which is to receive a therapeutic treatment. The tracking generator 26 is configured to emit tracking energy for use in connection with the first plurality of tracking elements (14,16,18) and the second plurality of tracking elements (20,22,24). In one embodiment, the tracking generator 26 comprises an electromagnetic field generator, wherein the generator is referenced to a fixed orientation and location, as indicated by reference numeral 27. Electromagnetic field generator generates an electromagnetic field in a region of interest, which includes a treatment region of the patient.

System controller 28 can comprise any suitable computer and/or sensor interfaces, the controller further being programmed with suitable instructions for carrying out the various functions as discussed herein with respect to performing therapeutic ultrasound tracking and treatment as discussed herein. System controller 28 may include various input/output signal lines, such as 38 and 44, for example, for being electronically coupling to other elements of the therapeutic ultrasound tracking system 10. A suitable display device 46 is coupled to system controller 28, for example, for use by a system operator during a given therapeutic ultrasound tracking and treatment application. Furthermore, additional devices, such as input/output devices, pointing devices, etc. (not shown) may be provided, as may be necessary, for a given implementation of therapeutic ultrasound tracking and treatment application. In addition, a means 48 for obtaining an image acquisition from storage (e.g., a memory or storage device containing previously obtained images from a given modality) or real-time image acquisition (e.g., real-time images from a given modality acquisition device) is coupled to system controller 28.

FIG. 2 is a schematic representation view of the therapeutic ultrasound probe 12 of FIG. 1 shown in greater detail for use with the system for positional tracking 10 of the present disclosure. In one embodiment, the tracking guide 40 is physically coupled to the therapeutic ultrasound probe 12. The tracking guide 40 can include, for example, a device display, wherein the display device produces visual representations of a plurality of visually concentric shapes (50,52,54), an indicator element 56, and directional indicators (58,60,62,64). The tracking guide 40 is coupled to the system controller 28, for example, via power and signal lines 38. The system controller 28 is adapted to control the tracking guide 40 to provide a guiding representation of position and orientation of the therapeutic ultrasound probe 12 with respect to the target region of the patient, wherein the target region of the patient is registered by the second plurality of tracking elements (20,22,24). The embodiments of the present disclosure thus advantageously allow for a more natural interaction (“freehand”) during a therapeutic treatment with the patient for the system operator or interventional radiologist. In addition, while the tracking guide 40 is illustrated and discussed as being coupled to the probe, the embodiments according to the present disclosure can include other implementations of the tracking guide, for example, implementations of the tracking guide as a portion of display 46 or other system component.

In one embodiment, the tracking guide 40 includes a plurality of visually concentric shapes (50,52,54) and an indicator element 56. The visually concentric shapes (50,52,54) represent various proximities of the therapeutic ultrasound probe 12 with respect to the target region. A location of the indicator element 56 with respect to the concentric shapes (50,52,54) provides the visual guiding representation of position and orientation of the therapeutic ultrasound probe 12 with respect to the target region. In one embodiment, the visually concentric shapes (50,52,54) can include one for indicating a proximity too close 50 in position to the target region, one for indicating a desired proximity in position 52 to the target region, and one for indicating a proximity too far 54 in position from the target region. While only three different sized shapes are illustrated and discussed with respect to concentric shapes (50,52,54), the embodiments according to the present disclosure can include more than three.

The indicator element 56 can be characterized by any number of different size shapes to convey a corresponding given proximity to the target region. For example, the indicator element 56 can be characterized by a first size shape, as illustrated in the rendition 70 of the tracking guide 40 in FIG. 2, for indicating a proximity too close in position to the target region. The indicator element 56 can be characterized by a second size shape, as illustrated in the renditions 68 and 72 of the tracking guide 40 in FIG. 2, for indicating a desired proximity in position to the target region. Note that in the rendition 68, the indicator element 56 is not centered, which is indicative that the probe is not yet in position and orientation with respect to the target region, as will be discussed further herein below. The indicator element 56 can also be characterized by a third size shape, as illustrated in the rendition 66 of the tracking guide 40 in FIG. 2, for indicating a proximity too far in position from the target region. While only three different sized shapes are illustrated and discussed with respect to indicator element 56, the embodiments according to the present disclosure can include more than three.

The tracking guide 40 further includes a plurality of directional indicators (58,60,62,64). The directional indicators (58,60,62,64) represent various orientations of therapeutic ultrasound probe movement needed with respect to the target region. Together with the visually concentric shapes (50,52,54) and the indicator element 56, the directional indicators (58,60,62,64) provide guidance for a system user to achieve the allowable position and orientation of the therapeutic ultrasound probe 12 with respect to the target region of the patient 42. The various orientations can include movement forward 58, movement backward 62, movement to the left 64 and movement to the right 60, as well as, movement in (as illustrated by indicator element 56 in rendition 66) and movement out (as illustrated by indicator element 56 in rendition 70), to best treat the intended region. In one embodiment, the guide includes a form of bubble which moves on/off center and grows/shrinks in size according to the relative movements needed. While only four different directional indicators (58,60,62,64) are illustrated and discussed with respect to directional indicators, the embodiments according to the present disclosure can include more or less than four.

FIG. 3 is a flow diagram view 80 illustrating positional tracking of a therapeutic ultrasound probe of the system and method according to an embodiment of the present disclosure. The method includes performing an initial placement of the patient and therapeutic probe in step 82. In step 84, location of the therapeutic probe is detected. In step 86, location of the patient and thus the treatment region (or zone) is detected. In step 88, a determination is made whether the probe is positioned to allow treatment. The determination is based at least upon positional tracking of the first plurality of tracking elements and the second plurality of tracking elements, as discussed herein. If it is determined that the probe is not positioned to allow treatment, then the method proceeds to step 90. In step 90, the system operator is prompted to move the probe in relationship to the patient, for example, as directed by the tracking guide 40. After step 90, the method returns to step 84 with detection of the location of the therapeutic probe as previously discussed. Referring again to step 88, if it is determined that the probe is positioned to allow treatment, then the process proceeds to step 92.

In step 92, a determination is made whether an acoustic path between the probe and the target region is blocked, for example, by bone or gas-filled cavities such as lungs. If it is determined that the acoustic path is blocked, then the method proceeds to step 94, otherwise the method proceeds to step 96. Step 94 includes performing an acoustic compensation for the probe. For example, acoustic compensation can include updating a power and beam angle of ultrasound delivery from the therapeutic ultrasound probe, the power and beam angle update being based at least upon tracking information as determined from the first plurality of tracking sensors, the second plurality of tracking sensors, and the tracking generator and prescribed requirements of the therapeutic treatment. Another example can include adjusting an aperture of the therapeutic ultrasound probe in response to (i) motion tracking of the therapeutic ultrasound probe and (ii) a blocking of an acoustic path of ultrasound delivery with respect to the target region of the patient, the aperture adjustment being based at least upon tracking information as determined from the first plurality of tracking sensors, the second plurality of tracking sensors, and the tracking generator and prescribed requirements of the therapeutic treatment. Another example includes adjusting ultrasound settings to compensate for an altered acoustic path to the target region. In the later instance, the adjustment of ultrasound settings is adapted to effectively compensate for voluntary and involuntary organ motion including respiration and heartbeat and for alteration of the acoustic path or movement of the target zone due to physical or biological response or consequences of the ultrasonic treatment. Still another example includes changing an electronic focusing of the therapeutic ultrasound probe in response to a detected therapeutic ultrasound probe location and a corresponding acoustic path to the target region. Upon a completion of acoustic compensation, the method proceeds to step 96.

In step 96, a representation of the treatment zone (or region) is provided on the display 46 (of FIG. 1). In particular, the representation of the treatment zone overlies a display of (i) a previously obtained image or previously obtained images or (ii) a real-time image or images. Following step 96, the therapeutic treatment is allowed to commence or to continue in step 98, as is appropriate. In step 100, a determination is made whether the desired therapeutic treatment is completed. If completed, then the method ends at step 102. If the desired therapeutic treatment is not yet completed, then the method returns to step 84 and thereafter proceeds as discussed herein above.

FIG. 4 is a partial block diagram view of a system 110 for positional tracking of a therapeutic ultrasound probe 112 according to another embodiment of the present disclosure. System 100 is similar to system 10 of FIG. 1, with the following differences. The therapeutic ultrasound tracking system 110 comprises a first plurality of tracking elements (114,116,118), a second plurality of tracking elements (120,122,124), a tracking generator 126, and a system controller 28. The first plurality of tracking elements (114,116,118) is disposed in a first position and orientation with respect to each another and with respect to an energy emission surface 30 of the therapeutic ultrasound probe 112. Therapeutic ultrasound probe 112 can include any suitable ultrasound probe that can be configured for implementing the embodiments of the present disclosure and for carrying out the requirements of a given ultrasound therapy. Therapeutic ultrasound probe 112 includes a housing portion 32 and a handle portion 34, which can be formed integral with one another. An ultrasound transducer 36 is disposed within housing portion 32, proximate surface 30, for emission of desired ultrasound energy. Various electrical power and signal lines, collectively represented by the feature indicated by reference numeral 38, are coupled between components of the therapeutic ultrasound probe 112 and system controller 28, for example, as appropriate, for carrying out various functions and steps described herein. A tracking guide 40 coupled to therapeutic ultrasound probe 112.

The second plurality of tracking elements (120,122,124) is adapted to be coupled to a patient 42 in a second position and orientation with respect to each other and with respect to a target region of the patient which is to receive a therapeutic treatment. The tracking generator 126 is configured to emit tracking energy for use in connection with the first plurality of tracking elements (114,116,118) and the second plurality of tracking elements (120,122,124). In one embodiment, the tracking generator 126 comprises an optical tracking generator, wherein the generator is referenced to a fixed orientation and location, as indicated by reference numeral 127. Optical tracking generator comprises, for example, an LED tracking generator. Optical tracking generator 126 generates optical signals in a region of interest, which includes a treatment region of the patient. In addition, the first plurality of tracking elements (114,116,118) and the second plurality of tracking elements (120,122,124) comprise optical detection sensors.

Accordingly, there has been disclosed a therapeutic ultrasound tracking system that includes a first and second plurality of tracking elements, a tracking generator, and a system controller. The first plurality of tracking elements is disposed in a first position and orientation with respect to each another and with respect to an energy emission surface of a therapeutic ultrasound probe. The second plurality of tracking elements is adapted to be coupled to a patient in a second position and orientation with respect to each other and with respect to a target region of the patient which is to receive a therapeutic treatment. The tracking generator emits tracking energy for use in connection with the first and second plurality of tracking elements. The system controller detects whether the probe is positioned within an allowable position and orientation with respect to the target region, and (i) responsive to detecting the probe being within the allowable position and orientation, the system controller enables an energization of the probe to perform one of (i)(a) commence the therapeutic treatment and (i)(b) continue the therapeutic treatment, and (ii) responsive to not detecting the probe being within the allowable position and orientation, the system controller disables the energization of the probe for the therapeutic treatment.

Commencement of the therapeutic treatment and continuation of the therapeutic treatment are according to prescribed requirements of the therapeutic treatment. The prescribed requirements can also include the system controller monitoring locations of the target region which have already received treatment and prohibiting excess treatment of the same locations beyond that required for the therapeutic treatment. In another embodiment, the second plurality of tracking elements is detectable in a medical image obtained via an ultrasound imaging system or an imaging system having a modality other than ultrasound. The system controller is further adapted to compute, in response to detection of the second plurality of tracking elements within the medical image, a relationship between (i) the target region in a coordinate system of the imaging system and (ii) the target region in coordinates of the tracking system. The controller is further adapted to automatically prescribe, in response to one or more measured positions of the therapeutic ultrasound probe with respect to the target region, one selected from the group consisting of: a CT, MR, PET and SPECT imaging scan in order to monitor a progress of the therapeutic treatment.

In one embodiment, the system controller is further configured to update a power and beam angle of ultrasound delivery from the therapeutic ultrasound probe in response to motion tracking of the therapeutic ultrasound probe with respect to the target region of the patient. The power and beam angle update is based at least upon tracking information as determined from the first plurality of tracking sensors, the second plurality of tracking sensors, and the tracking generator and prescribed requirements of the therapeutic treatment.

In another embodiment, the system controller is further configured to adjust an aperture of the therapeutic ultrasound probe in response to (i) motion tracking of the therapeutic ultrasound probe and (ii) a blocking of an acoustic path of ultrasound delivery with respect to the target region of the patient. The aperture adjustment is based at least upon tracking information as determined from the first plurality of tracking sensors, the second plurality of tracking sensors, and the tracking generator and prescribed requirements of the therapeutic treatment. In a further embodiment, the system controller is configured to adjust ultrasound settings of the therapeutic ultrasound probe in response to motion tracking of the therapeutic ultrasound probe with respect to the target zone of the patient. The adjusted ultrasound settings are configured to compensate for an altered acoustic path to the target zone. In a still further embodiment, the system controller is configured to change an electronic focusing of the therapeutic ultrasound probe in response to a detected therapeutic ultrasound probe location and a corresponding acoustic path to the target zone.

In one embodiment, the therapeutic treatment includes one or more of (i) ultrasound mediated drug delivery, (ii) ultrasound mediated gene delivery, (iii) ultrasound directed thermal therapies, (iv) high intensity focused ultrasound ablation, (v) sonothrombolysis, and (vi) ultrasound cosmetic surgery. In another embodiment, the first plurality of tracking elements comprises three tracking elements, and the second plurality of tracking elements comprises three tracking elements. In addition, the tracking generator can comprise at least one of (i) an electromagnetic field generator, wherein the first plurality and second plurality of tracking elements comprise electromagnetic sensors, or (ii) an optical tracking generator, wherein the first plurality and second plurality of tracking elements comprise optical detection sensors.

In another embodiment, a therapeutic ultrasound tracking system comprises a first plurality of tracking elements, a second plurality of tracking elements, a tracking generator, a system controller, and a tracking guide. The first plurality of tracking elements is disposed in a first position and orientation with respect to each another and with respect to an energy emission surface of a therapeutic ultrasound probe. The second plurality of tracking elements is adapted to be coupled to a patient in a second position and orientation with respect to each other and with respect to a target region of the patient which is to receive a therapeutic treatment. The tracking generator is configured to emit tracking energy for use in connection with the first plurality of tracking elements and the second plurality of tracking elements. In one embodiment, the tracking generator comprises one of (i) an electromagnetic field generator, wherein the first plurality and second plurality of tracking elements comprise electromagnetic sensors, or (ii) an optical tracking generator, wherein the first plurality and second plurality of tracking elements comprise optical detection sensors.

In the embodiment of the preceding paragraph, the system controller is coupled to the first plurality of tracking sensors, the second plurality of tracking sensors, and the tracking generator, and further adapted to be coupled to the therapeutic ultrasound probe. The system controller operates for detecting whether the therapeutic ultrasound probe is positioned within an allowable position and orientation with respect to the target region of the patient, and (i) responsive to detecting the therapeutic ultrasound probe being within the allowable position and orientation, the system controller enables an energization of the therapeutic ultrasound probe to perform one of (i)(a) commence the therapeutic treatment and (i)(b) continue the therapeutic treatment, and (ii) responsive to not detecting the therapeutic ultrasound probe being within the allowable position and orientation, the system controller disables the energization of the therapeutic ultrasound probe for the therapeutic treatment. The tracking guide is coupled to the system controller, the system controller further for controlling the tracking guide to provide a guiding representation of position and orientation of the therapeutic ultrasound probe with respect to the target region.

In addition, for the preceding embodiment, commencement of the therapeutic treatment and continuation of the therapeutic treatment are according to prescribed requirements of the therapeutic treatment. In addition, the prescribed requirements include the system controller monitoring locations of the target region which have already received treatment and prohibiting excess treatment of the same locations beyond that required for the therapeutic treatment.

According to another embodiment, a method for therapeutic ultrasound tracking comprises disposing a first plurality of tracking elements in a first position and orientation with respect to each another and with respect to an energy emission surface of a therapeutic ultrasound probe. A second plurality of tracking elements is coupled to a patient in a second position and orientation with respect to each other and with respect to a target region of the patient which is to receive a therapeutic treatment. Tracking energy is emitted from a tracking generator for use in connection with the first plurality of tracking elements and the second plurality of tracking elements. The method further includes detecting via a system controller whether the therapeutic ultrasound probe is positioned within an allowable position and orientation with respect to the target region of the patient. Responsive to (i) detecting the therapeutic ultrasound probe being within the allowable position and orientation, the method includes enabling an energization of the therapeutic ultrasound probe to perform one of (i)(a) commence the therapeutic treatment and (i)(b) continue the therapeutic treatment. Responsive to (ii) not detecting the therapeutic ultrasound probe being within the allowable position and orientation, the method includes disabling the energization of the therapeutic ultrasound probe for the therapeutic treatment. In one embodiment of the method, commencement of the therapeutic treatment and continuation of the therapeutic treatment are according to prescribed requirements of the therapeutic treatment. In addition, the prescribed requirements include monitoring locations of the target region which have already received treatment and prohibiting excess treatment of the same locations beyond that required for the therapeutic treatment.

In another embodiment, the method further includes updating via the system controller a power and beam angle of ultrasound delivery from the therapeutic ultrasound probe in response to motion tracking of the therapeutic ultrasound probe with respect to the target region of the patient. The power and beam angle updates are based at least upon tracking information as determined from the first plurality of tracking sensors, the second plurality of tracking sensors, and the tracking generator and prescribed requirements of the therapeutic treatment. In yet another embodiment, the method further includes adjusting via the system controller an aperture of the therapeutic ultrasound probe in response to (i) motion tracking of the therapeutic ultrasound probe and (ii) a blocking of an acoustic path of ultrasound delivery with respect to the target region of the patient. The aperture adjustment is based at least upon tracking information as determined from the first plurality of tracking sensors, the second plurality of tracking sensors, and the tracking generator and prescribed requirements of the therapeutic treatment. In another embodiment, the method further comprises controlling a tracking guide via the system controller to provide a guiding representation of position and orientation of the therapeutic ultrasound probe with respect to the target region.

Although only a few exemplary embodiments have been described in detail above, those skilled in the art will readily appreciate that many modifications are possible in the exemplary embodiments without materially departing from the novel teachings and advantages of the embodiments of the present disclosure. For example, the embodiments of the present disclosure can be applied to deposition of man-made devices, such as, nano-carriers, nano-beacons, nano-sensors, and nano-machines. Accordingly, all such modifications are intended to be included within the scope of the embodiments of the present disclosure as defined in the following claims. In the claims, means-plus-function clauses are intended to cover the structures described herein as performing the recited function and not only structural equivalents, but also equivalent structures.

In addition, any reference signs placed in parentheses in one or more claims shall not be construed as limiting the claims. The word “comprising” and “comprises,” and the like, does not exclude the presence of elements or steps other than those listed in any claim or the specification as a whole. The singular reference of an element does not exclude the plural references of such elements and vice-versa. One or more of the embodiments may be implemented by means of hardware comprising several distinct elements, and/or by means of a suitably programmed computer. In a device claim enumerating several means, several of these means may be embodied by one and the same item of hardware. The mere fact that certain measures are recited in mutually different dependent claims does not indicate that a combination of these measures cannot be used to an advantage.

1. A therapeutic ultrasound tracking system comprising:
a first plurality of tracking elements disposed in a first position and orientation with respect to each another and with respect to an energy emission surface of a therapeutic ultrasound probe;
a second plurality of tracking elements adapted to be coupled to a patient in a second position and orientation with respect to each other and with respect to a target region of the patient which is to receive a therapeutic treatment;
a tracking generator configured to emit tracking energy for use in connection with the first plurality of tracking elements and the second plurality of tracking elements; and
a system controller coupled to the first plurality of tracking sensors, the second plurality of tracking sensors, and the tracking generator, and further adapted to be coupled to the therapeutic ultrasound probe, the system controller for detecting whether the therapeutic ultrasound probe is positioned within an allowable position and orientation with respect to the target region of the patient, and (i) responsive to detecting the therapeutic ultrasound probe being within the allowable position and orientation, the system controller enables an energization of the therapeutic ultrasound probe to perform one of (i)(a) commence the therapeutic treatment and (i)(b) continue the therapeutic treatment, and (ii) responsive to not detecting the therapeutic ultrasound probe being within the allowable position and orientation, the system controller disables the energization of the therapeutic ultrasound probe for the therapeutic treatment.

2. The system of claim 1, wherein commencement of the therapeutic treatment and continuation of the therapeutic treatment are according to prescribed requirements of the therapeutic treatment.

3. The system of claim 2, further wherein the prescribed requirements include the system controller monitoring locations of the target region which have already received treatment and prohibiting excess treatment of the same locations beyond that required for the therapeutic treatment.

4. The system of claim 1, wherein the system controller is further configured to update a power and beam angle of ultrasound delivery from the therapeutic ultrasound probe in response to motion tracking of the therapeutic ultrasound probe with respect to the target region of the patient, the power and beam angle update being based at least upon tracking information as determined from the first plurality of tracking sensors, the second plurality of tracking sensors, and the tracking generator and prescribed requirements of the therapeutic treatment.

5. The system of claim 1, wherein the system controller is further configured to adjust an aperture of the therapeutic ultrasound probe in response to (i) motion tracking of the therapeutic ultrasound probe and (ii) a blocking of an acoustic path of ultrasound delivery with respect to the target region of the patient, the aperture adjustment being based at least upon tracking information as determined from the first plurality of tracking sensors, the second plurality of tracking sensors, and the tracking generator and prescribed requirements of the therapeutic treatment.

6. The system of claim 1, wherein the system controller is further configured to adjust ultrasound settings of the therapeutic ultrasound probe in response to motion tracking of the therapeutic ultrasound probe with respect to the target region of the patient, the adjusted ultrasound settings configured to compensate for an altered acoustic path to the target region.

7. The system of claim 1, further wherein the system controller is configured to change an electronic focusing of the therapeutic ultrasound probe in response to a detected therapeutic ultrasound probe location and a corresponding acoustic path to the target region.

8. The system of claim 1, further comprising:
a tracking guide coupled to the system controller, the system controller further for controlling the tracking guide to provide a guiding representation of position and orientation of the therapeutic ultrasound probe with respect to the target region.

9. The system of claim 8, wherein the tracking guide includes a plurality of visually concentric shapes and an indicator element, the visually concentric shapes representing various proximities of the therapeutic ultrasound probe with respect to the target region, and wherein a location of the indicator element with respect to the concentric shapes provides the visual guiding representation of position and orientation of the therapeutic ultrasound probe with respect to the target region.

10. The system of claim 9, further wherein the visually concentric shapes include one for indicating a proximity too close in position to the target region, one for indicating a desired proximity in position to the target region, and one for indicating a proximity too far in position from the target region.

11. The system of claim 9, wherein the indicator element is characterized by a first size shape for indicating a proximity too close in position to the target region, a second size shape for indicating a desired proximity in position to the target region, and a third size shape for indicating a proximity too far in position from the target region.

12. The system of claim 9, wherein the tracking guide further includes a plurality of directional indicators, the directional indicators representing various orientations of therapeutic ultrasound probe movement needed with respect to the target region, which together with the visually concentric shapes and the indicator element, provide guidance for a system user to achieve the allowable position and orientation of the therapeutic ultrasound probe with respect to the target region of the patient.

13. The system of claim 12, wherein the various orientations include movement forward, movement backward, movement to the left and movement to the right.

14. The system of claim 8, wherein the tracking guide is physically coupled to the therapeutic ultrasound probe.

15. The system of claim 14, wherein the tracking guide further includes a device display, and wherein the plurality of visually concentric shapes, indicator element, and directional indicators include visual representations on the device display.

16. The system of claim 1, wherein the therapeutic treatment includes at least one selected from the group consisting of (i) ultrasound mediated drug delivery, (ii) ultrasound mediated gene delivery, (iii) ultrasound directed thermal therapies, (iv) high intensity focused ultrasound ablation, (v) sonothrombolysis, and (vi) ultrasound cosmetic surgery.

17. The system of claim 1, wherein the first plurality of tracking elements comprise three tracking elements, and wherein the second plurality of tracking elements comprise three tracking elements.

18. The system of claim 1, wherein the tracking generator comprises one selected from the group consisting of (i) an electromagnetic field generator, wherein the first plurality and second plurality of tracking elements comprise electromagnetic sensors, and (ii) an optical tracking generator, wherein the first plurality and second plurality of tracking elements comprise optical detection sensors.

19. A therapeutic ultrasound tracking system comprising:
a first plurality of tracking elements disposed in a first position and orientation with respect to each another and with respect to an energy emission surface of a therapeutic ultrasound probe;
a second plurality of tracking elements adapted to be coupled to a patient in a second position and orientation with respect to each other and with respect to a target region of the patient which is to receive a therapeutic treatment;
a tracking generator configured to emit tracking energy for use in connection with the first plurality of tracking elements and the second plurality of tracking elements, wherein the tracking generator comprises one selected from the group consisting of (i) an electromagnetic field generator, wherein the first plurality and second plurality of tracking elements comprise electromagnetic sensors, and (ii) an optical tracking generator, wherein the first plurality and second plurality of tracking elements comprise optical detection sensors;
a system controller coupled to the first plurality of tracking sensors, the second plurality of tracking sensors, and the tracking generator, and further adapted to be coupled to the therapeutic ultrasound probe, the system controller for detecting whether the therapeutic ultrasound probe is positioned within an allowable position and orientation with respect to the target region of the patient, and (i) responsive to detecting the therapeutic ultrasound probe being within the allowable position and orientation, the system controller enables an energization of the therapeutic ultrasound probe to perform one of (i)(a) commence the therapeutic treatment and (i)(b) continue the therapeutic treatment, and (ii) responsive to not detecting the therapeutic ultrasound probe being within the allowable position and orientation, the system controller disables the energization of the therapeutic ultrasound probe for the therapeutic treatment; and
a tracking guide coupled to the system controller, the system controller further for controlling the tracking guide to provide a guiding representation of position and orientation of the therapeutic ultrasound probe with respect to the target region.

20. The system of claim 19, wherein commencement of the therapeutic treatment and continuation of the therapeutic treatment are according to prescribed requirements of the therapeutic treatment, further wherein the prescribed requirements include the system controller monitoring locations of the target region which have already received treatment and prohibiting excess treatment of the same locations beyond that required for the therapeutic treatment.

21. A method for therapeutic ultrasound tracking comprising:
disposing a first plurality of tracking elements in a first position and orientation with respect to each another and with respect to an energy emission surface of a therapeutic ultrasound probe;
coupling a second plurality of tracking elements to a patient in a second position and orientation with respect to each other and with respect to a target region of the patient which is to receive a therapeutic treatment;
emitting tracking energy from a tracking generator for use in connection with the first plurality of tracking elements and the second plurality of tracking elements; and
detecting via a system controller whether the therapeutic ultrasound probe is positioned within an allowable position and orientation with respect to the target region of the patient, and (i) responsive to detecting the therapeutic ultrasound probe being within the allowable position and orientation, enabling an energization of the therapeutic ultrasound probe to perform one of (i)(a) commence the therapeutic treatment and (i)(b) continue the therapeutic treatment, and (ii) responsive to not detecting the therapeutic ultrasound probe being within the allowable position and orientation, disabling the energization of the therapeutic ultrasound probe for the therapeutic treatment.

22. The method of claim 21, wherein commencement of the therapeutic treatment and continuation of the therapeutic treatment are according to prescribed requirements of the therapeutic treatment, further wherein the prescribed requirements include monitoring locations of the target region which have already received treatment and prohibiting excess treatment of the same locations beyond that required for the therapeutic treatment.

23. The method of claim 21, further including updating via the system controller a power and beam angle of ultrasound delivery from the therapeutic ultrasound probe in response to motion tracking of the therapeutic ultrasound probe with respect to the target region of the patient, the power and beam angle update being based at least upon tracking information as determined from the first plurality of tracking sensors, the second plurality of tracking sensors, and the tracking generator and prescribed requirements of the therapeutic treatment.

24. The method of claim 21, further including adjusting via the system controller an aperture of the therapeutic ultrasound probe in response to (i) motion tracking of the therapeutic ultrasound probe and (ii) a blocking of an acoustic path of ultrasound delivery with respect to the target region of the patient, the aperture adjustment being based at least upon tracking information as determined from the first plurality of tracking sensors, the second plurality of tracking sensors, and the tracking generator and prescribed requirements of the therapeutic treatment.

25. The method of claim 21, further comprising:
controlling a tracking guide via the system controller to provide a guiding representation of position and orientation of the therapeutic ultrasound probe with respect to the target region.

26. The system of claim 1, wherein the second plurality of tracking elements are detectable in a medical image obtained via an ultrasound imaging system or an imaging system having a modality other than ultrasound, and wherein the system controller is further adapted to compute, in response to detection of the second plurality of tracking elements within the medical image, a relationship between (i) the target region in a coordinate system of the imaging system and (ii) the target region in coordinates of the tracking system.

27. The system of claim 1, wherein the system controller is further adapted to automatically prescribe, in response to one or more measured positions of the therapeutic ultrasound probe with respect to the target region, one selected from the group consisting of: a CT, MR, PET and SPECT imaging scan in order to monitor a progress of the therapeutic treatment.
